(19) United States
(12) Patent Application Publication
(10) Pub. No.: US 2023/0241279 A1
(43) Pub. Date: Aug. 3, 2023

(54) AN ILLUMINATION SYSTEM COMPOSED OF AT LEAST ONE ILLUMINATION DEVICE AS WELL AS SUCH ILLUMINATION DEVICE

(71) Applicant: SIGNIFY HOLDING B.V., EINDHOVEN (NL)

(72) Inventors: RÉMY CYRILLE BROERSMA, EINDHOVEN (NL); KARS-MICHIEL HUBERT LENSSEN, VELDHOVEN (NL); MARC ANDRE DE SAMBER, LOMMEL (BE); WILLEM DIRK VAN DRIEL, 'S-HERTOGENBOSCH (NL)

(21) Appl. No.: 18/008,202

(22) PCT Filed: Jun. 1, 2021
(86) PCT No.: PCT/EP2021/064683

§ 371 Date: Dec. 5, 2022

(30) Foreign Application Priority Data
Jun. 3, 2020 (EP) 20178070.7
Sep. 28, 2020 (EP) 20198673.4

(51) Int. Cl. A61L 9/20; A61L 9/22

(52) U.S. Cl. CPC A61L 9/20; A61L 9/22; A61L 2209/111; A61L 2209/12

(57) ABSTRACT

The invention relates to an illumination system comprising at least one illumination device as well such illumination device, both the system as the device capable of implementing a lighting functionality as well as an air treatment (disinfecting or purifying) functionality. According to a first aspect of the invention an illumination system composed of at least one illumination device is proposed, with the at least one illumination device comprising a support structure; at least one light emitting source coupled with the support structure for emitting visible light radiation; an ion generating source coupled with the support structure for at least generating ionized molecules in air; and at least one UV light emitting source coupled with the support structure for emitting UV light radiation in a wavelength range for depleting ozone generated by the ion generating source. Herewith such illumination system can effectively implemented in free accessible spaces, whilst implementing both a lighting functionality as well as an air treatment (disinfecting or purifying) functionality and in addition also guaranteeing a safe, ozone free environment for people accessing such public spaces.

### TECHNICAL FIELD

The invention relates to an illumination system comprising at least one illumination device as well such illumination device, both the system as the device capable of implementing a lighting functionality as well as an air treatment (disinfecting or purifying) functionality.

### BACKGROUND OF THE INVENTION

There is a renewed interest in the application of UV to the treatment of indoor air and heating, ventilating and air conditioning (HVAC) equipment. UV treatment of air was popular in the 1950s and was utilized in areas such as food preparation and medical facilities.

It was particularly popular in the control of tuberculosis. Recently there has been a renewed interest in improving indoor air quality (IAQ) with UV. As with water, the public has become more concerned with the quality of its air. Many factors in our environment have put pressure on our air quality.
Tighter homes and buildings (i.e., less fresh air and ventilation).
Energy saving measures.
Decrease in the quality of outdoor air.
Substantial increases in the incidence of asthma.
Sick building syndrome.
The realization that mold (fungi) in the air can cause serious health problems within a building.

Basically, two applications of UV are becoming common. In one, the moving air stream is disinfected in much the same manner as with a water system. In the other application, stationary components of the system such as air conditioning coils, drain pans and filter surfaces are exposed to help prevent mold and bacteria growth or to disinfect the filter to aid in handling.

Common airborne virus and bacteria are readily deactivated with UV. Fungi (molds and spores) require much higher doses. Usually only the 254 nm wavelength UV is used in air as 185 nm produces ozone, which is undesirable in indoor air applications. Therefore, typically known ozone generating sources have the disadvantage that they cannot be used in a safe manner for air purification in spaces, which are freely accessible to humans.

Due to these health and safety concerns, in the consumer domain (single) air purifying devices are used of which some have ionizing generators included that can kill bacteria and viruses when air ion density is at the correct level.

Therefore, there is a need for an illumination system comprising at least one illumination device as well as such illumination device for use in free accessible spaces, such as office spaces, public spaces or e.g. supermarkets, with both the system as the device capable of implementing a lighting functionality as well as an air treatment (disinfecting or purifying) functionality, which furthermore mitigates any potential health hazards due to unwanted exposure to ozone.

US20120275960 discloses an illumination device comprising a visible light source, an ionizer, and a UV light emitting source.

### SUMMARY OF THE INVENTION

According to a first aspect of the invention an illumination system is proposed, with the at least one illumination device comprising a support structure; at least one light emitting source coupled with the support structure for emitting visible light radiation; an ion generating source coupled with the support structure for at least generating ionized molecules in air; and at least one UV light emitting source coupled with the support structure for emitting UV light radiation in a wavelength range for depleting ozone generated by the ion generating source.

Herewith such illumination system can effectively implemented in free accessible spaces, whilst implementing both a lighting functionality as well as an air treatment (disinfecting or purifying) functionality and in addition also guaranteeing a safe, ozone free environment for people accessing such public spaces.

In a preferred embodiment, the wavelength range of the UV light radiation is in the range of 220-360 nm, preferably between 240-320 nm.

Additionally the illumination system further comprises at least one detector for detecting an ozone concentration, whereas the at least one ozone concentration detector may be coupled with the support structure of the at least one illumination device.

In an improved example, a control device is present for operating the at least one light emitting source and/or the ion generating source and/or the at least one UV light emitting source in response to said ozone concentration being detected.

This provide accurate feedback about the ozone levels in the free accessible space, wherein the illumination system is installed and operating, and thus an effective depletion of ozone generated by the ion generating source.

The illumination system further comprises at least one housing accommodating the at least one light emitting source, the housing being coupled with the support structure further comprises a housing and being provided with an exit window for exiting at least the ionized molecules in air being generated by the ion generating source, and wherein the at least one UV light emitting source is mounted within the housing.

This construction prevents undesired and unhealthy UV radiation exposure of people in the free accessible space where the illumination system is installed.

In particular the exit window is configured as a labyrinth-shaped nozzle exit, thereby preventing direct (line-of-sight) leakage of harmful UV light radiation towards the space where the illumination system is installed.

In particular the at least one UV light emitting source may be mounted next to the exit window, effectively depleting ozone generated in the housing and thus preventing unwanted exiting of ozone from the housing.

The at least one UV light emitting source surrounds or is mounted in the direct proximity next to the ion generating source. Then, ozone generated in the housing is effectively depleted inside the housing. Furthermore, the housing near the exit window comprises an UV light radiation opaque material and in examples the UV light radiation opaque material is formed as an UV light radiation absorbing material or as a fluorescent material or a photocatalyst material. Herewith undesired and unhealthy UV radiation exposure is mitigated, in particular escaping of UV light radiation via reflections on the surface walls of the housing towards the fee accessible space. Furthermore, said UV opaque material can be transmissive for visible light, typically over the whole wavelength range of 400-700 nm, such that the illumination efficacy of the illumination device for visible light is not/hardly decreased.

In an example, the illumination system according to any one or more of the preceding claims, wherein the at least one UV light emitting device emits UV light radiation in a wavelength range for reducing airborne pathogens. This provides an additional sanitizing functionality of the air of the space where the illumination system is operated.

In a further embodiment the illumination system is formed of at least one illumination device, the at least one illumination device configured as an integrally formed unit comprising the support structure and the at least one light emitting source, the ion generating source and the at least one UV light emitting source coupled with the support structure.

In a further advantageous example of the disclosure, the at least one light emitting source and/or the at least one ion generating source and/or the at least one UV light emitting source are operatively interconnected in a data-communication network. In particular each of said operatively interconnected light emitting sources, ion generating sources and UV light emitting sources comprises a data-communication interface.

Alternatively, the illumination system is built of a plurality of said illumination devices, which are operatively interconnected in such a data-communications network.

Preferably the data-communications interfaces of each illumination device, or of each of said operatively interconnected light emitting sources, ion generating sources and UV light emitting sources operate in accordance with a network protocol for exchanging data between the networked illumination devices, such as designated ZigBee™, Bluetooth™, Wi-Fi based protocols for wireless networks, and wired bus networks such as DALI™ (Digital Addressable Lighting Interface), DSI (Digital Serial Interface), DMX (Digital Multiplex), KNX.

By implementing the various components such as each of said operatively interconnected light emitting sources, ion generating sources and UV light emitting sources and/or the multiple illumination devices according to the disclosure in a data-communication network, a more homogeneous coverage of the space where the illumination system is installed and operated with ionized air molecules is obtained.

Furthermore, the ozone depleting UV source may similarly be applied in various other types of apparatuses having a high voltage element that creates an electric field in air having and thus having the potential to create a corona discharge and hence to generate ozone, if the electric field value is high enough. Therefore the ozone destroying UV source may equally be applied in devices like power sources, transformers, printers, motors, radio transmitters, X-ray machines, etc., optionally equally provided with data-communications interfaces for interconnection to data-communications network. The electric potential gap that is necessary for triggering a corona discharge between two wires is defined by Peek's law, for example as explained in Wikipedia:

https://en.wikipedia.org/wiki/Peek%27s_law , referring to the original book by Peek (page 43), which is digitally available at:

https://archive.org/details/dielectricpheno00peekgoog/page/n190/mode/2up?q=corona+discharge. To give a rough indication: typically the electric field near the conductor should be in the order of 30 kV/cm or higher to create a corona discharge and on sharp points in air, corona discharges can start at voltages of about 2-6 kV, see for example at:

https://en.wikipedia.org/wiki/Coronadischarge.

### BRIEF DESCRIPTION OF THE DRAWINGS

The invention will now be discussed with reference to the drawings, which show in:

FIG. 1 schematically illustrates the Chapman mechanism for stratospheric ozone (O₂+O⇄O₃);

FIG. 2 a graph depicting the absorption characteristics of oxygen (O₂) and ozone (O₃)

FIG. 3 schematically illustrates details of an embodiment of an illumination device according to the present disclosure;

FIG. 4 schematically illustrates details of an embodiment of an illumination system implementing several illumination devices according to the present disclosure;

FIGS. 5, 6, 7 and 8 schematically illustrate another examples and details of embodiments of an illumination device according to the present disclosure.

### DETAILED DESCRIPTION OF THE INVENTION

For a proper understanding of the invention, in the detailed description below corresponding elements or parts of the invention will be denoted with identical reference numerals in the drawings.

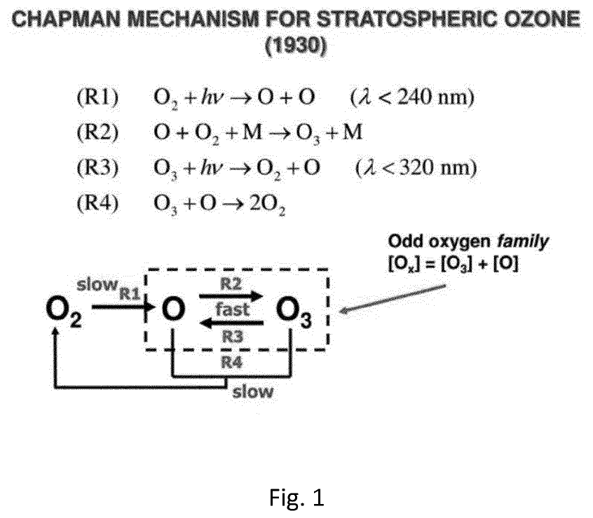

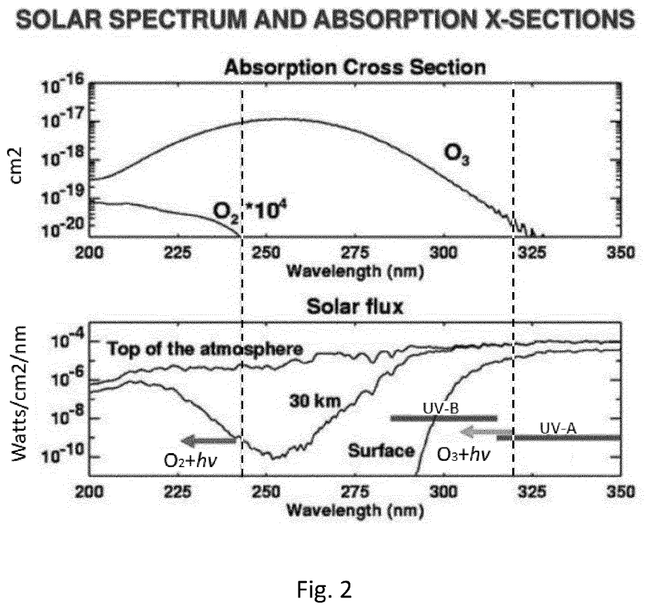

It is known in the prior art that ozone (O₃) can be created in the atmosphere by UV light radiation (partial reactions R1 and R2), but that O₃-molecules can also be destroyed or annihilated by UV light radiation according to partial reactions R3 and R4. This reaction cycle is known as the Chapman cycle as depicted in FIG. 1. The ozone creation and annihilation destruction depend on the absorption characteristics of oxygen (O₂) and ozone (O₃), as depicted in FIG. 2.

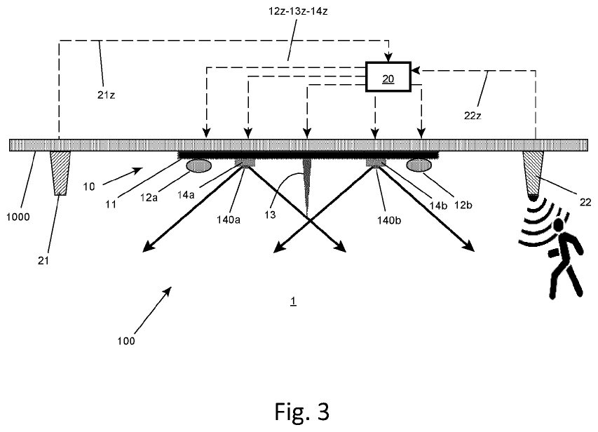

FIG. 3 schematically illustrate a non-limiting example of an embodiment of an illumination device according to the present disclosure. The illumination device is depicted with reference numeral 10 and is part of an illumination system 100. The illumination device 10 can be positioned at the lower part of a luminaire or at the top part of a luminaire, which luminaire can be mounted either on the ground floor or at the ceiling 1000 (as shown in FIG. 3) of a free accessible space 1, such as office spaces, or public spaces, such as theatre's, cinema's or supermarkets.

The illumination device 10 of the illumination system 100 comprises a support structure 11. The support structure 11 may be part of a housing 15 of the illumination device 10 or luminaire. The support structure 11 may contain or comprise electric circuitry and electric components for providing electric power to the several components of the illumination device 10.

One of those functional components of the illumination device 10 is depicted with reference numerals 12a and 12b, being a light emitting source. The light emitting sources 12a-12b are coupled with the support structure and are arranged for emitting light radiation in the visible wavelength range. Although in the Figures the examples of the illumination device 10 are shown with two light emitting sources 12a-12b, it is noted that other example might incorporated a single light emitting source 12a or more than two light emitting sources.

The light emitting sources 12a-12b can be configured as any type of light source, either LEDs, incandescent light bulbs, fluorescent beam lights, etc. etc. Another functional component of the illumination device 10 is an ion generating source 13. The ion generating source 13 is also mounted or coupled with the support structure 11 and designed for at least generating ionized molecules in air. The ion generating source 13 can be a negative ion generator or a Chizhevsky's chandelier. It uses a high voltage to ionize (electrically charge) air molecules. Negative ions, or anions, are particles with one or more extra electrons, conferring a net negative charge to the particle.

Cations are positive ions missing one or more electrons, resulting in a net positive charge.

This ionization principle can be used in free accessible spaces, such as office spaces, which are regularly occupied by persons. Every room or space 1 is filled with positively charged particles, which could be made up of dust, microbes, odours, airborne bacteria or illnesses, smoke or other allergens. The negatively charged particles attract and bond to the positively charged particles in the space 1 or room making them too heavy to be airborne. The use of negative ions in the air can have the following results:

Improved air quality through the removal of dust, allergens, pollen, pet dander, inactivation of viruses, mould spores and other airborne bacteria;
Decreased exposure to airborne pathogens (viruses, bacteria, molds) that can cause respiratory symptoms like for instance colds, flu and asthmatic triggers;
Improved sleep and improving mood and treating mood disorders;
Relief from seasonal or chronic depression, negative ions can have as much of an effect as prescribed antidepressants.

Due to these health and safety concerns, in the consumer domain (single) air purifying devices are used of which some have ionizing generators included that can kill bacteria and viruses when air ion density is at the correct level.

Accordingly, the illumination device 10 (and the illumination system 100) is arranged to implement a lighting functionality by means of the light emitting source (or sources) 12a-12b as well as an air treatment (disinfecting or purifying) functionality with the ion generating source 13.

As a side effect, ion generating sources 13 are known for generating—next to ionized air molecules—also ozone molecules (O₃). Ozone is beneficial when present in the stratosphere, approx. at 10-50 kilometres high, but ozone is harmful at ground level, where such ion generating sources or ionizers typically are used. Hence, the ozone levels in a building space might vary widely depending on setup of the ionizer system. However, ion generating sources also generating ozone have the disadvantage that they cannot be used in a safe manner for air purification in spaces, which are freely accessible to humans. As high ozone concentrations are harmful to humans, this constitutes a formidable challenge to bring luminaire-integrated ionizers to the mass market.

Therefore, there is a need for an illumination system comprising at least one illumination device as well as such illumination device for use in free accessible spaces, such as office spaces, public spaces or e.g. supermarkets, with both the system as the device capable of implementing a lighting functionality as well as an air treatment (disinfecting or purifying) functionality, which furthermore mitigates any potential health hazards due to unwanted exposure to ozone.

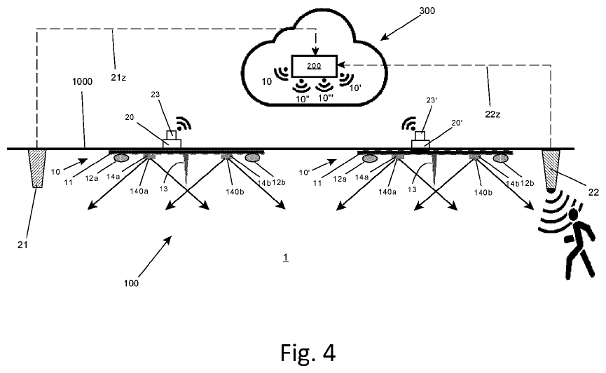

Accordingly, in the example of FIGS. 3 and 4, at least one UV light emitting source 14a-14b is coupled with the support structure 11. The UV light emitting source 14a-14b, when activated, emit UV light radiation in a wavelength range, which wavelength is effective in depleting ozone generated by the ion generating source 13. Each UV light emitting source 14a-14b is provided with an UV radiation emitting surface 140a-140b. Although in the Figures the examples of the illumination device 10 are shown with two UV light emitting source 14a-14b, it is noted that other examples might incorporated a single UV light emitting source 14a or more than two UV light emitting sources.

Herewith such illumination system can effectively implemented in free accessible spaces, whilst implementing both a lighting functionality as well as an air treatment (disinfecting or purifying) functionality and in addition also guaranteeing a safe, ozone free environment for people accessing such public spaces. The effective wavelength range of the UV light radiation for depleting ozone generated by the ion generating source 13 is in the range of 220-360 nm, preferably between 240-320 nm, most preferably between 240-280 nm where ozone has the largest response of UV radiation for ozone depletion. Preferably the UV light emitting sources 14a-14b are mounted to the support structure 11, next to the ion generating device 13. In a particular embodiment both UV light emitting sources 14a-14b surround or are mounted in the direct proximity next to the ion generating source 13. Alternatively, the UV light emitting sources can be embodied as a single UV light emitting source.

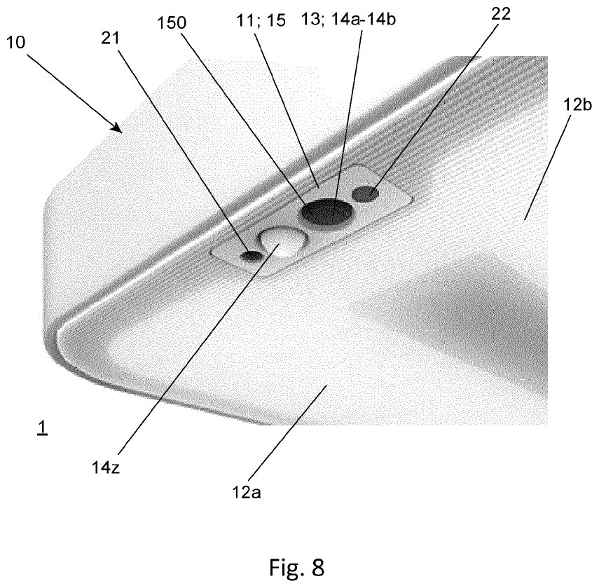

In another advantageous example, the UV light emitting sources 14a-14b may perform a dual UV function of firstly emitting UV light radiation in the effective wavelength range for depleting or annihilating (destroying) ozone molecules generated by the ion generating source 13 and secondly providing upper-air purification (e.g. to kill airborne viruses) through emitting UV light radiation with another wavelength range. This embodiment is e.g. shown in FIG. 8, showing another example of a (suspended) luminaire or illumination device 10 according to the invention.

The illumination device 10 contains two light emitting sources 12a-12b as well as an ion generating source 13 and the UV light emitting sources 14a-14b for ozone depletion. A further UV light emitting source 14z can be used for upper air purification by emitting UV light radiation in a wavelength range, which wavelength range is effective in reducing airborne pathogens. This provides an additional sanitizing functionality of the air of the space 1 where the illumination system is operated.

It is noted that both functionalities of ozone depletion and upper air purification can be combined in one UV light emitting source (for example the UV light emitting source 14z), which can be controlled to emit UV light radiation in different, desired wavelength ranges, depending on the desired functionality.

The advantage of integrated ion generating source (ionizers) 13 in suspended luminaires 10 as depicted in FIG. 8, is that the ionizers 13 are located at closer proximity towards humans and possible contaminated surfaces. A second benefit is that the ionizers 13 and detectors 21 can be mounted at the top part of the luminaire/illumination device 10 as enough space is available. This also ensures that the ozone concentration is measured where it matters for human safety (i.e. close to the head) instead of at ceiling level 1000. The ozone detectors 21 and UV LED's 14a-14b can also be easily added to the top part of the luminaire/illumination device 10, providing ozone reduction as well as using the UV lighting additionally for upper air disinfection.

Spacers around the edges of the luminaire 10 can prevent UV light from being directed towards humans. FIG. 8 shows space for ionizers, detectors and UV light sources in suspended luminaires, for example at bottom and/or at the top of the luminaire. In an example as shown in FIGS. 3 and 4 the UV radiation emitting surfaces 140a-140b of the UV light emitting sources 14a-14b are directed towards the open, free accessible space 1 or room, resulting in the UV light radiation being emitted to react with and deplete or annihilate ozone molecules, which are present in the air and for upper air purification, as example above.

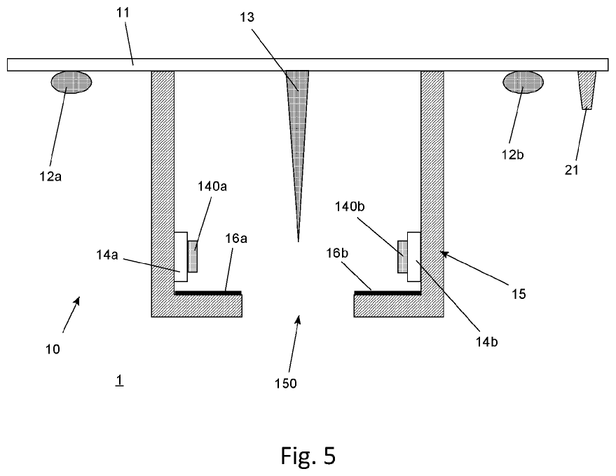

In another embodiments, as shown in FIGS. 5, 6 and 7, the illumination system 10 comprises a housing 15, which is mounted to or is part of the support structure 11. In the housing 15 both the UV light emitting sources 14a-14b as well as the ion generating source 13 are integrated. The housing 15 is provided with an exit window 150 for exiting at least the ionized molecules in the air of the space 1 or room where the illumination system is installed and operated. Alternatively, also one or all light emitting sources 12a-12b can be integrated in the housing 15 as shown in FIG. 8.

In an example of the housing 15 being mounted to the support structure 11, the housing 15 may be detachable and can be coupled and decoupled from the support structure 11 by means of suitable interacting coupling means present on both the housing 15 and the support structure 11. The interacting coupling means allow—when the housing 15 is coupled and mounted to the support structure 11—for both a mechanical coupling as well as an electrical coupling between the illumination system 100 and the several components mounted inside the housing 15. The housing 15 can be shaped as lighting fixture.

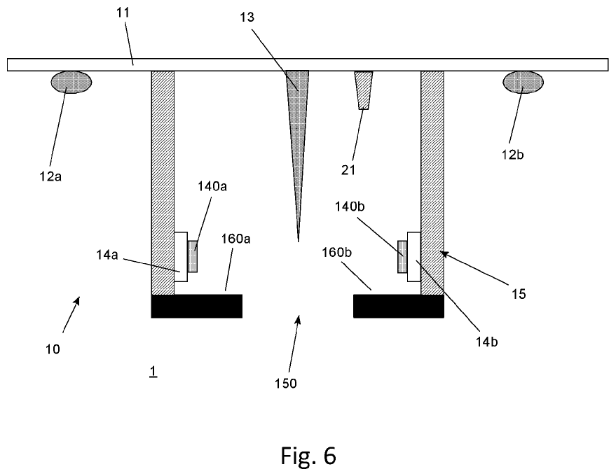

As in FIGS. 3 and 4, also in the embodiments of FIGS. 5-7 the UV light emitting sources 14a-14b are mounted to a housing wall inside the housing 15 and in the direct proximity of the ion generating source 13. In FIGS. 3 and 4 the UV radiation emitting surfaces 140a-140b of the UV light emitting sources 14a-14b are directed towards the open, free accessible space 1 or room, whereas in in FIGS. 5 and 6 the UV radiation emitting surfaces 140a-140b are pointed or directed towards the ion generating source 13.

As the UV light emitting sources 14a-14b are mounted close to or next to the exit window 150 of the housing, ozone generated in the housing 15 is effectively depleted inside the housing 15. Any unwanted exiting of ozone from the housing 15 is thus prevented.

Additionally, this construction prevents undesired and unhealthy UV radiation exposure of people in the free accessible space 1 where the illumination system is installed.

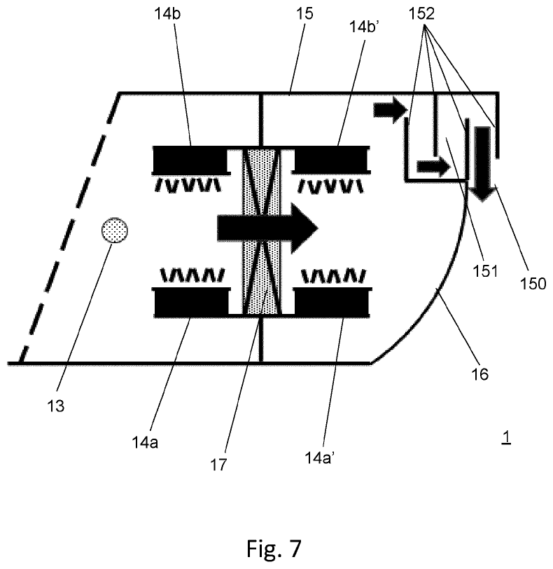

To further prevent any exiting of harmful UV light radiation from the housing 15 towards the fee accessible space 1, the exit window can be configured as a labyrinth-shaped nozzle exit as shown in the embodiment of FIG. 7. In FIG. 7 the exit windows 150 is configured as an nozzle exit 151, which is not straight but has a labyrinth configuration due to several wall sections 152. Herewith any preventing direct (line-of-sight) leakage of harmful UV light radiation towards the space 1 where the illumination system 100 is installed, is prevented.

The housing 15 of the embodiment of FIG. 7 also contains a fan 17 for forcefully creating an air flow through the housing 15 towards the labyrinth-shaped nozzle exit 150-151. The air flow also contains ionized air molecules generated by the ion generating source 13. Any ozone molecules generated by the ion generating source 13 are effectively depleted or annihilated by several UV light emitting sources 14a-14b/14a′-14b′ being mounted in either or both an upstream or downstream location relative to the fan 17, as seen in the air flow direction of the air flow through the housing 15.

As an additional prevention of unwanted leakage of harmful UV light radiation from the housing 15, the housing 15 near the exit window 150 may comprise an UV light radiation opaque material. The UV light radiation opaque material can be incorporated in wall parts next to the housing window 150 as an UV light radiation absorbing or blocking dope, as denoted with reference numerals 160a and 160b in FIG. 6. In another example, as depicted in the embodiment of FIGS. 5 and 7 the UV light radiation opaque material is formed as an UV light radiation absorbing material or as a fluorescent material or a photocatalyst material, which is applied as a layer 16-16a-16b to, preferably the inner surface wall of the housing 15 directly next to the housing window 150.

With these embodiments of FIGS. 5-7 undesired and unhealthy UV radiation exposure is mitigated, in particular escaping of UV light radiation via reflections on the surface walls of the housing 150 towards the fee accessible space 1.

In the shown embodiments of FIGS. 3-8 UV light radiation emitting sources 14a-14b/14a′-14b′ can be configured as UVC/UVB LED's devices, emitting in the wavelength range of 220-360 nm, preferably between 240-320 nm to deplete or destroy ozone molecules. In an example a UV light radiation wavelength of 254 nm can be used, this wavelength is very efficiently generated by a low pressure mercury discharge lamp device, to deplete or destroy ozone without implementing any UV shielding 16-16a-16b-160a-160b.

As a further improvement, reference numeral 21 denotes an ozone concentration detector 21 for detecting an ozone concentration in the space 1 or room where the illumination system 100 is installed. The ozone concentration detector 21 may be coupled with the support structure 11 of at least one illumination device 10, as shown in FIGS. 5 and 6, whereas in the latter embodiment of FIG. 6 the ozone concentration detector 21 is mounted inside the housing 15. Alternatively, as shown in FIGS. 3 and 4, the ozone concentration detector 21 of the illumination system 100 is provided at an arbitrary location within the space 1 or room where the illumination system 100 is installed, for example at the ceiling 1000 of the room 1.

Although in the Figures the examples of the illumination device 10 (illumination system 100) are shown with one ozone concentration detector 21 positioned in the space 1 to be illuminated and monitored, it is noted that other examples might incorporated more than one ozone concentration detector 21, which are located at different, distinct and distant positions (for example in different housings 15 of the illumination devices 10-10′ being part of the illumination system) from each other in the same space or room 1 to be illuminated and monitored.

In either embodiments, the ozone concentration detector (or detectors) 21 detects an ozone concentration level and generates a detection signal (indicated with 21z in FIGS. 3 and 4) in response to that. Sensing ozone levels and triggering alarms 21z based on these detections combined with ozone depleting UV light radiation emitting sources provide a valuable asset for reducing ozone background levels in offices and schools.

For an effective control, in an example as shown in FIG. 3, the illumination system 100 comprises a control device 20, capable of operating—by generating and issuing proper control signals (indicated with 12z-13z-14z in FIG. 3)—the at least one light emitting source 12a-12b and/or the ion generating source 13 and/or the at least one UV light emitting source 14a-14b of each illumination device 10 being part of the illumination system 100. These control signals 12z-13z-14z are send to the respective light emitting sources, the ion generating sources and/or the UV light radiation emitting sources of each illumination device 10 in response to said ozone concentration 21z being detected by the ozone concentration detector (or detectors) 21.

In yet another embodiment an ozone concentration detector 21 may be positioned outside the space 1 or room to be illuminated and monitored in order to detect an outdoor ozone concentration, based on which detection the UV light radiation emitting sources 14a-14b/14a′-14b′ are controlled.

This provides accurate feedback about the ozone levels in the free accessible space 1, where the illumination system 100 is installed and operating, and thus an effective depletion of ozone generated by the ion generating source 13.

In particular, proper activation control signals 14z need only be send to and activate the UV light radiation emitting sources 14a-14b/14a′-14b′ of the respective illumination devices 10 in the event that ozone levels detected in the building space 1 become too high.

In a similar manner the control device 20 can generate and send proper activation control signals 13z only to the ion generating source 13 of a respective illumination device 10 in order to regulate the level of disinfection ionization (and possibly adjusts the operating mode (e.g. voltage, frequency) of the ion generating source 13) depending on the measured ozone values.

Similarly, the control device 20 can generate and send control signals to the fan 17 (FIG. 7) to control the air flow through the housing 15 of the illumination device 10 depending on the measured ozone values.

As an additional feature, the illumination system 100 may comprise one or more human detection sensors 22 (for example IR sensors) mounted to the ceiling 1000 or a wall of the space 1 for detecting the presence or absence of humans in the space 1 or room to be illuminated and monitored. The human detection signal 22z thus generated by the human detection sensor(s) 22 and forwarded to the control device 20 can be used to e.g. switch on (or off) the light emitting sources 12a-12b and likewise to generate proper control signals 13z and 14z for controlling, such as activating or deactivating, the ion generating source 13 and/or the at least one UV light emitting source 14a-14b of each illumination device 10.

In a further embodiment the illumination system 100 is shown in FIG. 4, wherein the illumination system 100 is built of a plurality of illumination devices 10-10′-10″-10″′-etc.-etc. The plurality of illumination devices 10-10′-10″-10″′ -etc. are in this embodiment mounted to the ceiling 1000 of a space 1 to be illuminated and monitored and are operatively interconnected in a data-communication network 300. As depicted in this example, the illumination devices 10-10′ as depicted individually comprise a support structure 11. On the support structure 11 the at least one light emitting source 12a-12b, the ion generating source 13 and the UV light radiation emitting sources 14a-14b are integrally mounted, each illumination device 10-10′ thus forming a single unit. Additionally, the support structure 11 of each single unit illumination device 10-10′ can be part of a housing, as depicted in FIG. 8.

As an additional feature of the example of FIG. 4, each of said operatively interconnected illumination devices 10-10′-10″ comprises a data-communication interface 23-23′-23″ as well as a control device 20-20′-20″ for controlling the several components of each individual illumination device 10-10′-10″-10″′ as detailed above.

Alternatively, either of the light emitting sources 12a-12b, ion generating sources 13 and UV light emitting sources 14a-14b may each comprise such a data-communication interface 23, resulting in the at least one light emitting source 12a-12b and/or the at least one ion generating source 13 and/or the at least one UV light emitting source 14a-14b being operatively interconnected, and thus forming a data-communication network.

The data-communications interfaces 23-23′-23″-23″′-etc. of either the operatively interconnected illumination devices 10-10′-10″ or of the several light emitting sources 12a-12b, ion generating sources 13 and UV light emitting sources 14a-14b operate together within the data-communication network 300 in accordance with a network protocol for exchanging data between the networked illumination devices, such as designated ZigBee™, Bluetooth™, Wi-Fi based protocols for wireless networks (depicted with the wireless icon in FIG. 4). Alternatively, wired bus networks such as DALI™ (Digital Addressable Lighting Interface), DSI (Digital Serial Interface), DMX (Digital Multiplex), KNX can be implemented.

The illumination system 100 operates a central control device 200, e.g. a server in the cloud, in the data-communication network 300. The central control device 200 receives the several detection signals 21z-22z-etc. generated by the ozone concentration detectors 21 and human detection sensors 22 and processes these signals in order to generate and send proper control signals (via wireless or wired network transmission) to the data-communications interfaces 23-23′-23″-23″′-etc. of either the operatively interconnected illumination devices 10-10′-10″ or of the several operatively interconnected light emitting sources 12a-12b, ion generating sources 13 and UV light emitting sources 14a-14b and subsequently to the control devices 20-20′-20″-20″′-etc. for proper control (activation, de-activation, etc.) of the several components, such as the light emitting sources 12a-12b, ion generating source 13, UV light radiation emitting sources 14a-14b-14a′-14b′, and the fan 17.

By implementing an illumination system 100 composed of multiple interconnected illumination devices 10-10′-10″-10″′ in a data-communication network 300, a more homogeneous coverage of the space 1 where the illumination system 100 is installed and operated with ionized air molecules, is obtained.

1. An illumination system comprising:
a support structure;
at least one light emitting source coupled with the support structure for emitting visible light radiation;
an ion generating source coupled with the support structure for at least generating ionized molecules in air; and
at least one UV light emitting source coupled with the support structure for emitting UV light radiation in a wavelength range for depleting ozone generated by the ion generating source,
wherein the illumination system further comprises at least one housing accommodating the at least one light emitting source, the housing being coupled with the support structure and being provided with an exit window for exiting at least the ionized molecules in air being generated by the ion generating source, and wherein the at least one UV light emitting source is mounted within the housing,
wherein the at least one UV light emitting source surrounds or is mounted in the direct proximity next to the ion generating source, and
wherein the housing prevents exiting UV radiation from the housing to freely accessible space, in that the housing near the exit window comprises an UV light radiation opaque material.

2. The illumination system according to claim 1, wherein the wavelength range of the UV light radiation is in the range of 220-360 nm, preferably between 240-320 nm.

3. The illumination system according to claim 1, wherein the illumination system further comprises at least one detector for detecting an ozone concentration, the at least one ozone concentration detector being coupled with the support structure.

4. The illumination system according to claim 3, further comprising a control device for operating the at least one light emitting source and/or the ion generating source and/or the at least one UV light emitting source in response to said ozone concentration being detected.

5. The illumination system according to claim 1, wherein the exit window is configured as a labyrinth exit.

6. The illumination system according to claim 1, wherein the at least one UV light emitting source is mounted next to the exit window.

7. The illumination system according to claim 1, wherein the UV light radiation opaque material is transmissive for visible light in the wavelength range of 400-700 nm.

8. The illumination system according to claim 1, wherein the UV light radiation opaque material is formed as an UV light radiation absorbing material or as a fluorescent material or a photocatalyst material.

9. The illumination system according to claim 1, wherein the at least one UV light emitting source emits UV light radiation in a wavelength range for reducing airborne pathogens.

10. The illumination system according to claim 1, wherein the illumination system is formed of at least one illumination device, the at least one illumination device configured as an integrally formed unit comprising the support structure and the at least one light emitting source, the ion generating source and the at least one UV light emitting source coupled with the support structure.

11. The illumination system according to claim 1, wherein the at least one light emitting source and/or the at least one ion generating source and/or the at least one UV light emitting source are operatively interconnected in a data-communication network.

12. The illumination system according to claim 11, wherein each of said operatively interconnected light emitting sources, ion generating sources and UV light emitting sources comprise a data-communication interface.

13. The illumination system according to claim 12, wherein the data-communication interface operates in accordance with a network protocol for exchanging data between the networked illumination devices, such as designated ZigBee™, Bluetooth™, Wi-Fi based protocols for wireless networks, and wired bus networks such as DALI™ (Digital Addressable Lighting Interface), DSI (Digital Serial Interface), DMX (Digital Multiplex), KNX.
